(19) United States
(12) Patent Application Publication
(10) Pub. No.: US 2013/0144028 A1
(43) Pub. Date: Jun. 6, 2013

(54) PROCESSES FOR PRODUCING DIAMINOBUTANE (DAB), SUCCINIC DINITRILE (SDN) AND SUCCINAMIDE (DAM)

(71) Applicants: Olan S. Fruchey, Hurricane, WV (US); Leo E. Manzer, Wilmington, DE (US); Dilum Dunuwila, Princeton, NJ (US); Brian T. Keen, Pinch, WV (US); Brooke A. Albin, Charleston, WV (US); Nye A. Clinton, Hurricane, WV (US); Bernard D. Dombek, Charleston, WV (US)

(72) Inventors: Olan S. Fruchey, Hurricane, WV (US); Leo E. Manzer, Wilmington, DE (US); Dilum Dunuwila, Princeton, NJ (US); Brian T. Keen, Pinch, WV (US); Brooke A. Albin, Charleston, WV (US); Nye A. Clinton, Hurricane, WV (US); Bernard D. Dombek, Charleston, WV (US)

(73) Assignee: BIOAMBER S.A.S., Bazancourt (FR)

(21) Appl. No.: 13/697,645

(22) PCT Filed: May 17, 2011
(86) PCT No.: PCT/US11/36755

§ 371 Date: Jan. 9, 2013

Related U.S. Application Data
(60) Provisional application No. 61/346,164, filed on May 19, 2010.

(30) Foreign Application Priority Data
May 19, 2010 (US) 61346164

(51) Int. Cl. C07C 253/22; C07C 253/20; C07C 209/48; C07C 253/30; C07C 209/46; C07C 231/02

(52) U.S. Cl. CPC C07C 253/22; C07C 209/46; C07C 231/02; C07C 209/48; C07C 253/30; C07C 253/20
USPC 528/335; 564/468; 558/311; 564/138; 564/491; 558/459; 564/490; 528/362

(57) ABSTRACT

Processes include providing a clarified diammonium succinate (DAS)- or monoammonium succinate (MAS)- containing fermentation broth; distilling the broth of an overhead that includes water and ammonia, and a liquid bottoms that includes SA, and at least about 20 wt % water; cooling the bottoms to a temperature sufficient to cause the bottoms to separate into a liquid portion in contact with a solid portion that is substantially pure SA; separating the solid portion from the liquid portion; and converting the solid portion to produce nitrogen containing compounds such as diamino butane (DAB), succinic dinitrile (SDN), succinic amino nitrile (SAN) or succinamide (DAM) and downstream products.

### RELATED APPLICATION

This application claims the benefit of U.S. Provisional Application No. 61/346,164, filed May 19, 2010, the subject matter of which is hereby incorporated by reference.

### TECHNICAL FIELD

This disclosure relates to processes for producing DAB, SDN and DAM from succinic acid (SA) produced by fermentation as well as downstream products.

### BACKGROUND

Certain carbonaceous products of sugar fermentation are seen as replacements for petroleum-derived materials for use as feedstocks for the manufacture of carbon-containing chemicals. One such product is monoammonium succinate (MAS).

A material related to MAS, namely SA, can be produced by microorganisms using fermentable carbon sources such as sugars as starting materials. However, most commercially viable, succinate producing microorganisms described in the literature neutralize the fermentation broth to maintain an appropriate pH for maximum growth, conversion and productivity. Typically, the pH of the fermentation broth is maintained at or near a pH of 7 by introduction of ammonium hydroxide into the broth, thereby converting the SA to diammonium succinate (DAS). The DAS must be converted to MAS to derive MAS from the fermentation broth.

Kushiki (Japanese Published Patent Application, Publication No. 2005-139156) discloses a method of obtaining MAS from an aqueous solution of DAS that could be obtained from a fermentation broth to which an ammonium salt is added as a counter ion. Specifically, MAS is crystallized from an aqueous solution of DAS by adding acetic acid to the solution to adjust the pH of the solution to a value between 4.6 and 6.3, causing impure MAS to crystallize from the solution.

Masuda (Japanese Unexamined Application Publication P2007-254354, Oct. 4, 2007) describes partial deammoniation of dilute aqueous solutions of “ammonium succinate” of the formula H₄NOOCCH₂CH₂COONH₄. From the molecular formula disclosed, it can be seen that “ammonium succinate” is diammonium succinate. Masuda removes water and ammonia by heating solutions of the ammonium succinate to yield a solid SA-based composition containing, in addition to ammonium succinate, at least one of monoammonium succinate, succinic acid, monoamide succinate, succinimide, succinamide or ester succinate. Thus, it can be inferred that like Kushiki, Masuda discloses a process that results in production of impure MAS. The processes of both Kushiki and Masuda lead to materials that need to be subjected to various purification regimes to produce high purity MAS.

Bio-derived SA such as that derived from MAS and/or DAS is a platform molecule for synthesis of a number of commercially important chemicals and polymers. Therefore, it is highly desirable to provide a technology that offers flexibility to integrate clear, commercially viable paths from SA to derivatives such as DAB, SDN and DAM and further downstream products. In response to the lack of an economically and technically viable process solution for converting fermentation-derived SA to DAB, SDN, succinic amino nitrile (SAN) and DAM, it could be helpful to provide methods for providing a cost effective SA stream of sufficient purity for direct hydrogenation.

### SUMMARY

We provide a process for making nitrogen containing compounds including providing a clarified DAS-containing fermentation broth; distilling the broth under super atmospheric pressure at a temperature of greater than 100° C. to about 250° C. to form an overhead that includes water and ammonia, and a liquid bottoms that includes SA, and at least about 20 wt % water; cooling the bottoms to a temperature sufficient to cause the bottoms to separate into a liquid portion in contact with a solid portion that is substantially pure SA; separating the solid portion from the liquid portion; (1) contacting the solid portion with hydrogen and ammonia in the presence of at least one hydrogenation catalyst to produce DAB; or (2) dehydrating at least part of the solid portion to produce SDN; or (3) dehydrating at least part of the solid portion to produce DAM; and recovering the DAB, SDN or DAM.

We also provide a process for making nitrogen containing compounds including providing a clarified DAS-containing fermentation broth; adding an ammonia separating solvent to the broth; distilling the broth at a temperature and pressure sufficient to form an overhead that includes water and ammonia, and a liquid bottoms that includes SA, and at least about 20 wt % water; cooling the bottoms to a temperature sufficient to cause the bottoms to separate into a liquid portion in contact with a solid portion that is substantially pure SA; separating at least part of the solid portion from the liquid portion; (1) contacting the solid portion with hydrogen and ammonia in the presence of at least one hydrogenation catalyst to produce DAB; or (2) dehydrating at least part of the solid portion to produce SDN; or (3) dehydrating at least part of the solid portion to produce DAM; and recovering the DAB, SDN or DAM.

We further provide a process for making nitrogen containing compounds including providing a clarified MAS-containing fermentation broth; distilling the broth under super atmospheric pressure at a temperature of greater than 100° C. to about 250° C. to form an overhead that includes water and ammonia, and a liquid bottoms that includes SA, and at least about 20 wt % water; cooling the bottoms to a temperature sufficient to cause the bottoms to separate into a liquid portion in contact with a solid portion that is substantially pure SA; separating at least part of the solid portion from the liquid portion; (1) contacting the solid portion with hydrogen and ammonia in the presence of at least one hydrogenation catalyst to produce DAB; or (2) dehydrating at least part of the solid portion to produce SDN; or (3) dehydrating at least part of the solid portion to produce DAM; and recovering the DAB, SDN or DAM.

We further yet provide a process for making nitrogen containing compounds including providing a clarified MAS-containing fermentation broth; adding an ammonia separating solvent to the broth; distilling the broth at a temperature and pressure sufficient to form an overhead that includes water and ammonia, and a liquid bottoms that includes SA, and at least about 20 wt % water; cooling the bottoms to a temperature sufficient to cause the bottoms to separate into a liquid portion in contact with a solid portion that is substantially pure SA; separating at least part of the solid portion from the liquid portion; (1) contacting the solid portion with hydrogen and ammonia in the presence of at least one hydrogenation catalyst to produce DAB; or (2) dehydrating at least part of the solid portion to produce SDN; or (3) dehydrating at least part of the solid portion to produce DAM; and recovering the DAB, SDN or DAM.

### BRIEF DESCRIPTION OF THE DRAWINGS

FIG. 1 schematically illustrates a fully integrated process for producing fermentation-derived SA and subsequently converting SA to DAB, SDN and DAM.

FIG. 2 schematically illustrates a portion of FIG. 1, shown in greater detail, that provides selected reaction pathways from SA to DAB, SDN and DAM and other selected downstream products.

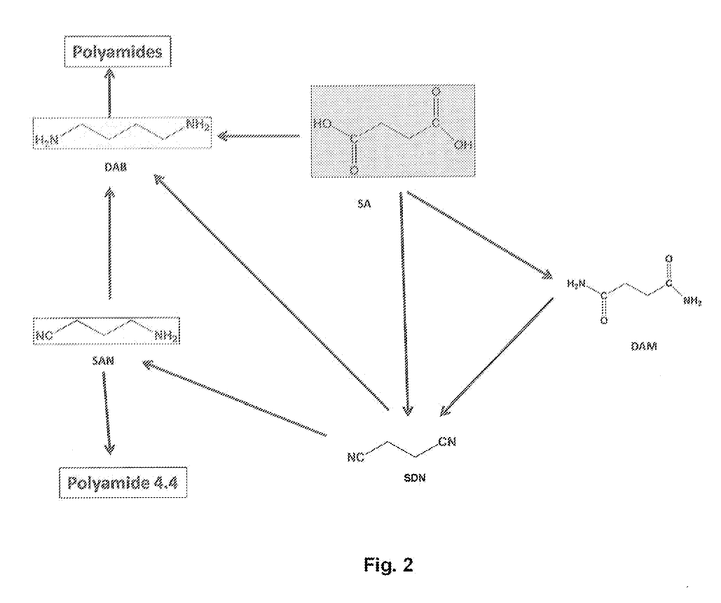

FIG. 3 is a graph showing the solubility of SA as a function of temperature in both water and a 20 wt % aqueous MAS solution.

### DETAILED DESCRIPTION

It will be appreciated that at least a portion of the following description is intended to refer to representative examples of processes selected for illustration in the drawings and is not intended to define or limit the disclosure, other than in the appended claims.

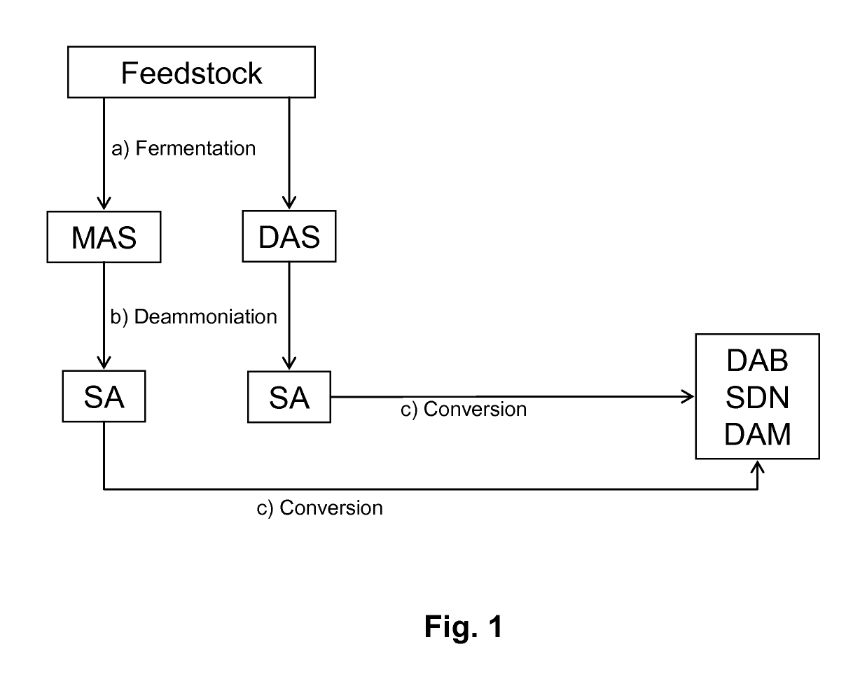

Our processes may be appreciated by reference to FIG. 1, which shows in flow diagram form one representative example of a bioprocessing system/process.

A growth vessel, typically an in-place steam sterilizable fermentor, may be used to grow a microbial culture that is subsequently utilized for the production of the DAS- containing fermentation broth. Such growth vessels are known in the art and are not further discussed.

The microbial culture may comprise microorganisms capable of producing succinic acids from fermentable carbon sources such as carbohydrate sugars. Representative examples of microorganisms include Escherichia coli (E. coli), Aspergillus niger, Corynebacterium glutamicum (also called Brevibacterium flavum), Enterococcus faecalis, Veillonella parvula, Actinobacillus succinogenes, Mannheimia succiniciproducens, Anaerobiospirillum succiniciproducens, Paecilomyces Varioti, Saccharomyces cerevisiae, Bacteroides fragilis, Bacteroides ruminicola, Bacteroides amylophilus, Alcaligenes eutrophus, Brevibacterium ammoniagenes, Brevibacterium lactofermentum, Candida brumptii, Candida catenulate, Candida mycoderma, Candida zeylanoides, Candida paludigena, Candida sonorensis, Candida utilis, Candida zeylanoides, Debaryomyces hansenii, Fusarium oxysporum, Humicola lanuginosa, Kloeckera apiculata, Kluyveromyces lactis, Kluyveromyces wickerhamii, Penicillium simplicissimum, Pichia anomala, Pichia besseyi, Pichia media, Pichia guilliermondii, Pichia inositovora, Pichia stipidis, Saccharomyces bayanus, Schizosaccharomyces pombe, Torulopsis candida, Yarrowia lipolytica, mixtures thereof and the like.

A preferred microorganism is an E. coli strain deposited at the ATCC under accession number PTA-5132. More preferred is this strain with its three antibiotic resistance genes (cat, amphl, tetA) removed. Removal of the antibiotic resistance genes cat (coding for the resistance to chloramphenicol), and amphl (coding for the resistance to kanamycin) can be performed by the so-called “Lambda-red (7k-red)” procedure as described in Datsenko K A and Wanner B L., Proc. Natl. Acad. Sci. USA 2000 Jun. 6; 97(12) 6640-5, the subject matter of which is incorporated herein by reference. The tetracycline resistant gene tetA can be removed using the procedure originally described by Bochner et al., J Bacteriol. 1980 August; 143(2): 926-933, the subject matter of which is incorporated herein by reference. Glucose is a preferred fermentable carbon source for this microorganism.

A fermentable carbon source (e.g., carbohydrates and sugars), optionally a source of nitrogen and complex nutrients (e.g., corn steep liquor), additional media components such as vitamins, salts and other materials that can improve cellular growth and/or product formation, and water may be fed to the growth vessel for growth and sustenance of the microbial culture. Typically, the microbial culture is grown under aerobic conditions provided by sparging an oxygen-rich gas (e.g., air or the like). Typically, an acid (e.g., sulphuric acid or the like) and ammonium hydroxide are provided for pH control during the growth of the microbial culture.

In one example, the aerobic conditions in the growth vessel (provided by sparging an oxygen-rich gas) are switched to anaerobic conditions by changing the oxygen-rich gas to an oxygen-deficient gas (e.g., CO₂ or the like). The anaerobic environment triggers bioconversion of the fermentable carbon source to succinic acid in situ in the growth vessel. Ammonium hydroxide is provided for pH control during bioconversion of the fermentable carbon source to SA. The SA that is produced is at least partially if not totally neutralized to DAS due to the presence of the ammonium hydroxide, leading to the production of a broth comprising DAS. The CO₂ provides an additional source of carbon for the production of SA.

In another example, the contents of the growth vessel may be transferred via a stream to a separate bioconversion vessel for bioconversion of a carbohydrate source to SA. An oxygen-deficient gas (e.g., CO₂ or the like) is sparged in the bioconversion vessel to provide anaerobic conditions that trigger production of SA. Ammonium hydroxide is provided for pH control during bioconversion of the carbohydrate source to SA. Due to the presence of the ammonium hydroxide, the SA produced is at least partially neutralized to DAS, leading to production of a broth that comprises DAS. The CO₂ provides an additional source of carbon for production of SA.

In another example, the bioconversion may be conducted at relatively low pH (e.g., 3-6). A base (ammonium hydroxide or ammonia) may be provided for pH control during bioconversion of the carbohydrate source to SA. Depending of the desired pH, due to the presence or lack of the ammonium hydroxide, either SA is produced or the SA produced is at least partially neutralized to MAS, DAS, or a mixture comprising SA, MAS and/or DAS. Thus, the SA produced during bioconversion can be subsequently neutralized, optionally in an additional step, by providing either ammonia or ammonium hydroxide leading to a broth comprising DAS. As a consequence, a “DAS-containing fermentation broth” generally means that the fermentation broth comprises DAS and possibly any number of other components such as MAS and/or SA, whether added and/or produced by bioconversion or otherwise. Similarly, a “MAS-containing fermentation broth” generally means that the fermentation broth comprises MAS and possibly any number of other components such as DAS and/or SA, whether added and/or produced by bioconversion or otherwise.

The broth resulting from the bioconversion of the fermentable carbon source (in either the growth vessel or the bioconversion vessel, depending on where the bioconversion takes place), typically contains insoluble solids such as cellular biomass and other suspended material, which are transferred via a stream to a clarification apparatus before distillation. Removal of insoluble solids clarifies the broth. This reduces or prevents fouling of subsequent distillation equipment. The insoluble solids can be removed by any one of several solid-liquid separation techniques, alone or in combination, including but not limited to, centrifugation and filtration (including, but not limited to ultra-filtration, micro-filtration or depth filtration). The choice of filtration technique can be made using techniques known in the art. Soluble inorganic compounds can be removed by any number of known methods such as but not limited to ion-exchange, physical adsorption and the like.

An example of centrifugation is a continuous disc stack centrifuge. It may be useful to add a polishing filtration step following centrifugation such as dead-end or cross-flow filtration, which may include the use of a filter aide such as diatomaceous earth or the like, or more preferably ultra-filtration or micro-filtration. The ultra-filtration or micro-filtration membrane can be ceramic or polymeric, for example. One example of a polymeric membrane is SelRO MPS-U2OP (pH stable ultra-filtration membrane) manufactured by Koch Membrane Systems (850 Main Street, Wilmington, Mass., USA). This is a commercially available polyethersulfone membrane with a 25,000 Dalton molecular weight cut-off which typically operates at pressures of 0.35 to 1.38 MPa (maximum pressure of 1.55 MPa) and at temperatures up to 50° C. Alternatively, a filtration step may be employed, such as ultra-filtration or micro-filtration alone.

The resulting clarified DAS-containing broth or MAS-containing broth, substantially free of the microbial culture and other solids, is transferred via a stream to a distillation apparatus.

The clarified distillation broth should contain DAS and/or MAS in an amount that is at least a majority of, preferably at least about 70 wt %, more preferably 80 wt % and most preferably at least about 90 wt % of all the diammonium dicarboxylate salts in the broth. The concentration of DAS and/or MAS as a weight percent (wt %) of the total dicarboxylic acid salts in the fermentation broth can be easily determined by high pressure liquid chromatography (HPLC) or other known means.

Water and ammonia are removed from the distillation apparatus as an overhead, and at least a portion is optionally recycled via a stream to the bioconversion vessel (or the growth vessel operated in the anaerobic mode).

Distillation temperature and pressure are not critical as long as the distillation is carried out in a way that ensures that the distillation overhead contains water and ammonia, and the distillation bottoms comprises at least some MAS and at least about 20 wt % water. A more preferred amount of water is at least about 30 wt % and an even more preferred amount is at least about 40 wt %. The rate of ammonia removal from the distillation step increases with increasing temperature and also can be increased by injecting steam during distillation. The rate of ammonia removal during distillation may also be increased by conducting distillation under a vacuum or by sparging the distillation apparatus with a non-reactive gas such as air, nitrogen or the like.

Removal of water during the distillation step can be enhanced by the use of an organic azeotroping agent such as toluene, xylene, cyclohexane, methyl cyclohexane, methyl isobutyl ketone, heptane or the like, provided that the bottoms contains at least about 20 wt % water. If the distillation is carried out in the presence of an organic agent capable of forming an azeotrope consisting of the water and the agent, distillation produces a biphasic bottoms that comprises an aqueous phase and an organic phase, in which case the aqueous phase can be separated from the organic phase, and the aqueous phase used as the distillation bottoms. Byproducts such as succinamide and succinimide are substantially avoided provided the water level in the bottoms is maintained at a level of at least about 30 wt %.

A preferred temperature for the distillation step is in the range of about 50° C. to about 300° C., depending on the pressure. A more preferred temperature range is about 150° C. to about 240° C., depending on the pressure. A distillation temperature of about 170° C. to about 230° C. is preferred. “Distillation temperature” refers to the temperature of the bottoms (for batch distillations this may be the temperature at the time when the last desired amount of overhead is taken).

Adding a water miscible organic solvent or an ammonia separating solvent facilitates deammoniation over a variety of distillation temperatures and pressures as discussed above. Such solvents include aprotic, bipolar, oxygen-containing solvents that may be able to form passive hydrogen bonds. Examples include, but are not limited to, diglyme, triglyme, tetraglyme, sulfoxides such as dimethylsulfoxide (DMSO), amides such as dimethylformamide (DMF) and dimethylacetamide, sulfones such as dimethylsulfone, gamma-butyrolactone (GBL), sulfolane, polyethyleneglycol (PEG), butoxytriglycol, N-methylpyrolidone (NMP), ethers such as dioxane, methyl ethyl ketone (MEK) and the like. Such solvents aid in the removal of ammonia from the DAS or MAS in the clarified broth. Regardless of the distillation technique, it is important that the distillation be carried out in a way that ensures that at least some MAS and at least about 20 wt % water remain in the bottoms and even more advantageously at least about 30 wt %. The distillation can be performed at atmospheric, sub-atmospheric or super-atmospheric pressures.

Under other conditions such as when the distillation is conducted in the absence of an azeotropic agent or ammonia separating solvent, the distillation is conducted at super atmospheric pressure at a temperature of greater than 100° C. to about 300° C. to form an overhead that comprises water and ammonia, and a liquid bottoms that comprises SA and at least about 20 wt % water. Super atmospheric pressure typically falls within a range of greater than ambient atmosphere up to and including about 25 atmospheres. Advantageously the amount of water is at least about 30 wt %.

The distillation can be a one-stage flash, a multistage distillation (i.e., a multistage column distillation) or the like. The one-stage flash can be conducted in any type of flasher (e.g., a wiped film evaporator, thin film evaporator, thermosiphon flasher, forced circulation flasher and the like). The multistages of the distillation column can be achieved by using trays, packing or the like. The packing can be random packing (e.g., Raschig rings, Pall rings, Berl saddles and the like) or structured packing (e.g., Koch-Sulzer packing, Intalox packing, Mellapak and the like). The trays can be of any design (e.g., sieve trays, valve trays, bubble-cap trays and the like). The distillation can be performed with any number of theoretical stages.

If the distillation apparatus is a column, the configuration is not particularly critical, and the column can be designed using well known criteria. The column can be operated in either stripping mode, rectifying mode or fractionation mode. Distillation can be conducted in either batch, semi-continuous or continuous mode. In the continuous mode, the broth is fed continuously into the distillation apparatus, and the overhead and bottoms are continuously removed from the apparatus as they are formed. The distillate from distillation is an ammonia/water solution, and the distillation bottoms is a liquid, aqueous solution of MAS and SA, which may also contain other fermentation by-product salts (i.e., ammonium acetate, ammonium formate, ammonium lactate and the like) and color bodies.

The distillation bottoms can be transferred via a stream to a cooling apparatus and cooled by conventional techniques. Cooling technique is not critical. A heat exchanger (with heat recovery) can be used. A flash vaporization cooler can be used to cool the bottoms to about 15° C. Cooling to 15° C. typically employs a refrigerated coolant such as, for example, glycol solution or, less preferably, brine. A concentration step can be included prior to cooling to help increase product yield. Further, both concentration and cooling can be combined using known methods such as vacuum evaporation and heat removal using integrated cooling jackets and/or external heat exchangers.

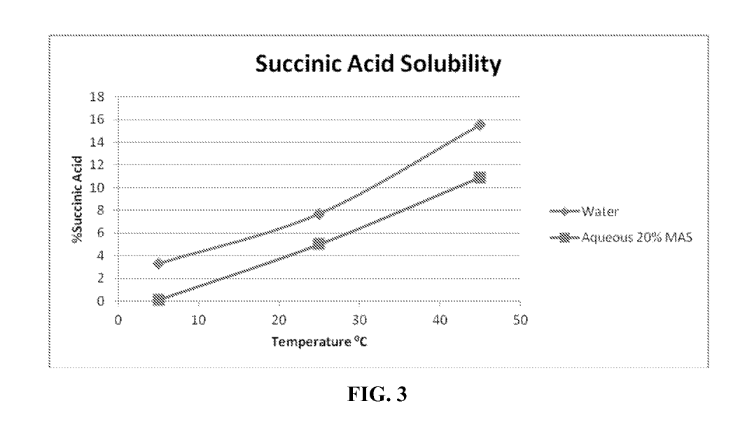

We found that the presence of some MAS in the liquid bottoms facilitates cooling-induced separation of the bottoms into a liquid portion in contact with a solid portion that at least “consists essentially” of SA (meaning that the solid portion is at least substantially pure crystalline SA) by reducing the solubility of SA in the liquid, aqueous, MAS-containing bottoms. FIG. 3 illustrates the reduced solubility of SA in an aqueous 20 wt % MAS solution at various temperatures ranging from 5° C. to 45° C. We discovered, therefore, that SA can be more completely crystallized out of an aqueous solution if some MAS is also present in that solution. A preferred concentration of MAS in such a solution is about 20 wt % or higher. This phenomenon allows crystallization of SA (i.e., formation of the solid portion of the distillation bottoms) at temperatures higher than those that would be required in the absence of MAS.

The distillation bottoms, after cooling, is fed via a stream to a separator for separation of the solid portion from the liquid portion. Separation can be accomplished via pressure filtration (e.g., using Nutsche or Rosenmond type pressure filters), centrifugation and the like. The resulting solid product can be recovered as product and dried, if desired, by standard methods.

After separation, it may be desirable to treat the solid portion to ensure that no liquid portion remains on the surface(s) of the solid portion. One way to minimize the amount of liquid portion that remains on the surface of the solid portion is to wash the separated solid portion with water and dry the resulting washed solid portion. A convenient way to wash the solid portion is to use a so-called “basket centrifuge.” Suitable basket centrifuges are available from The Western States Machine Company (Hamilton, Ohio, USA).

The liquid portion of the distillation bottoms (i.e., the mother liquor) may contain remaining dissolved SA, any unconverted MAS, any fermentation byproducts such as ammonium acetate, lactate, or formate, and other minor impurities. This liquid portion can be fed via a stream to a downstream apparatus. In one instance, the downstream apparatus may be a means for making a de-icer by treating in the mixture with an appropriate amount of potassium hydroxide, for example, to convert the ammonium salts to potassium salts. Ammonia generated in this reaction can be recovered for reuse in the bioconversion vessel (or the growth vessel operating in the anaerobic mode). The resulting mixture of potassium salts is valuable as a de-icer and anti-icer.

The mother liquor from the solids separation step can be recycled (or partially recycled) to a distillation apparatus via a stream to further enhance recovery of SA, as well as further convert MAS to SA.

The solid portion of the cooling-induced crystallization is substantially pure SA and is, therefore, useful for the known utilities of SA.

HPLC can be used to detect the presence of nitrogen-containing impurities such as succinamide and succinimide. The purity of SA can be determined by elemental carbon and nitrogen analysis. An ammonia electrode can be used to determine a crude approximation of SA purity.

Depending on the circumstances and various operating inputs, there are instances when the fermentation broth may be a clarified MAS-containing fermentation broth or a clarified SA-containing fermentation broth. In those circumstances, it can be advantageous to add MAS, DAS and/or SA and, optionally, ammonia, and/or ammonium hydroxide to those fermentation broths to facilitate the production of substantially pure SA. For example, the operating pH of the fermentation broth may be oriented such that the broth is a MAS-containing broth or a SA-containing broth. MAS, DAS, SA, ammonia, and/or ammonium hydroxide may be optionally added to those broths to attain a broth pH preferably less than about 6 to facilitate production of the above-mentioned substantially pure SA. In one particular form, it is especially advantageous to recycle SA, MAS and water from the liquid bottoms resulting from the distillation step, and/or the liquid portion from the liquid/solid separator, into the fermentation broth and/or clarified fermentation broth. In referring to the MAS-containing broth, such broth generally means that the fermentation broth comprises MAS and possibly any number of other components such as DAS and/or SA, whether added and/or produced by bioconversion or otherwise.

Streams comprising SA, MAS and/or DAS as described above may be converted to selected downstream products such as nitrogen containing compounds including but not limited to DAB, SDN, SAN, DAM and the like as described below. In initiating those processes, typically the SA, MAS and/or DAS may be dissolved in water to form an aqueous solution thereof which can be directly fed to the downstream reactor.

The SA, MAS or DAS may be converted to SDN, either directly or indirectly through the intermediate DAM by dehydration. Such dehydrations may be achieved thermally, enzymatically or in the presence of catalysts. Thus, appropriate temperatures, pressures and catalysts are selected to achieve the appropriate level of dehydration, depending on whether the conversion to SDN occurs directly or indirectly.

For example, the conversion should employ an appropriate dehydrating catalyst such as acidic or basic catalysts, including phosphates as disclosed in U.S. Pat. No. 4,237,067 and supported catalysts utilizing Ti, V, Hf or Zr on clays or alumina as disclosed is U.S. Pat. No. 5,587,498. Such catalysts are typically employed at temperatures of about 220° C. to about 350° C. at pressures of about 170 to 600 psig, for example.

Alternatively, dehydration can be achieved thermally as disclosed in U.S. Pat. No. 3,296,303, wherein acids plus an ammonia source are thermally dehydrated in the presence of glycol solvents at temperatures of 100° C. to 130° C. at pressures of 150 to 200 psig.

As a consequence, SA, MAS or DAS may be dehydrated directly to SDN or indirectly to SDN by the intermediate DAM. Then, once SDN is produced, it is possible to convert SDN directly to an amine such as DAB or to indirectly convert SDN to DAB through the intermediate SAN.

For example, direct conversion from SDN to DAB can be achieved in any number of ways such as disclosed in U.S. Pat. No. 6,376,714, wherein dinitriles in the presence of hydrogen and an ammonia source are converted utilizing catalysts such as Fe, Co, Ni, Rh or Pd promoted with Ru, Cr or W at temperatures of 50° C. to 150° C. at 300 to 1500 psig. The result is high yields of the diamine, in this case DAB.

Similarly, U.S. Pat. No. 4,003,933 converts nitriles to amines with hydrogen over a Co/ZrO₂ catalyst at 120° C. to 130° C. and at 1500 psig. Other catalysts may include Fe, Rh, Ir and Pt on TiO₂ or ZrO₂.

The indirect conversion of SDN to SAN can be achieved by selecting appropriate hydrogenation conditions such as those disclosed in U.S. Pat. No. 5,151,543, wherein nitriles are converted to amino nitriles, in this case SDN to SAN, utilizing RANEY catalysts such as Co or Ni promoted with Fe, Cr or Mo with hydrogen and an ammonia source at 50° C. to 80° C. at pressures of 250 to 1000 psig.

Similarly, the amino nitrile or diamino compounds can be co-produced from dinitriles such as those disclosed in U.S. Pat. No. 7,132,562. US '562 utilizes Fe, Co, Ru, Ni catalysts modified with Cr, V, Ti or Mn at temperatures of 50° C. to 250° C. and 3000 to 5000 psig to achieve high yields and selectivity to the diamine or amino nitrile. The catalysts may also be modified with ordinary P or N with HCN, or CO and hydrogen and an ammonia source.

It is also possible to convert SA, MAS or DAS directly to diamines such as DAB directly or indirectly through DAM. For example, U.S. Pat. No. 2,223,303 discloses the conversion of acids to amines with hydrogen and an ammonia source or alkyl amines with a Cd or Cu catalyst at temperatures of 200° C. to 450° C. at pressures of 10 to 300 ATM. Similarly, U.S. Pat. No. 3,579,583 discloses the conversion of dicarboxylic acids to amines, particularly alkyl amines, utilizing hydrogen and an ammonia source at temperatures of 200° C. to 300° C. at pressures of 100 to 300 ATM in the presence of a Zn—Al₂O₃ or Zn—Cr catalyst.

Further, U.S. Pat. No. 4,935,546 discloses the conversion of acids to amines with hydrogen and an ammonia source in the presence of a Co, Cu or Cr catalyst on a TiO₂ or Al₂O₃ support at temperatures of 250° C. to 350° C. and at pressures of 20 to 150 bar.

Once the conversions to DAB and SAN have been completed, it is also possible to convert those compounds into polyamide-type compounds in any number of ways known in the art. Representative examples include the following conversions. Polyamides may be produced from amino nitriles such as SAN. One example of conversions of this type may be found in U.S. Pat. No. 5,109,104 which converts an omega amino nitrile in the presence of an oxygenated phosphorus catalyst with water. This is generally achieved in a several-step conversion at temperatures of 200° C. to 330° C. and at pressures ranging from 250 to 350 psig.

An alternative methodology is disclosed in U.S. Pat. No. 6,958,381, wherein a starting monomer such as SAN may be polymerized into a polyamide in the presence of a chain regulator containing a nitrile group and a functional group capable of forming a carboxamide group.

Polyamides may also be formed from the diamines such as DAB, wherein the DAB is polymerized with a dicarboxylic acid or ester to form the polyamide. The preferred dicarboxylic acids have a chain length of C₄ to C₁₂. The dicarboxylic acid or ester may be an aromatic dicarboxylic acid or ester or it may be an alkyl dicarboxylic acid.

The subject matter and contents of the above-mentioned U.S. Pat. Nos. 4,237,067; 5,587,498; 3,296,303; 6,376,714; 4,003,933; 5,151,543; 7,132,562; 2,223,303; 3,579,583; 4,935,546; 5,109,104; and 6,958,381 are incorporated herein by reference.

### EXAMPLES

Our processes are illustrated by the following non-limiting representative examples. In Examples 1 and 2, a synthetic, aqueous DAS solution was used in place of an actual clarified DAS-containing fermentation broth.

The use of a synthetic DAS solution is believed to be a good model for the behavior of an actual broth in our processes because of the solubility of the typical fermentation by-products found in actual broth. The major by-products produced during fermentation are ammonium acetate, ammonium lactate and ammonium formate. If these impurities are present during the distillation step, one would not expect them to lose ammonia and form free acids in significant quantities until all of the DAS had been converted to SA. This is because acetic acid, lactic acid and formic acid are stronger acids than the second acid group of SA (pKa=5.48). In other words, acetate, lactate, formate and even monohydrogen succinate are weaker bases than the dianion succinate. Furthermore, ammonium acetate, ammonium lactate and ammonium formate are significantly more soluble in water than SA, and each is typically present in the broth at less than 10% of the DAS concentration. In addition, even if the acids (acetic, formic and lactic acids) were formed during the distillation step, they are miscible with water and will not crystallize from water. This means that the SA reaches saturation and crystallizes from solution (i.e., forming the solid portion), leaving the acid impurities dissolved in the mother liquor (i.e., the liquid portion).

### Example 1

This experiment shows the conversion of DAS to SA in an aqueous media.

An experiment was conducted in a 300 ml Hastelloy C stirred Parr reactor using a 15% (1.0 M) synthetic DAS solution. The reactor was charged with 200 g of solution and pressurized to 200 psig. The contents were then heated to begin distillation, bringing the temperature to approximately 200° C. Ammonia and water vapor were condensed overhead with cooling water and collected in a reservoir. Fresh water was pumped back to the system at a rate equal to the make rate (approximately 2 g/min) to maintain a constant succinate concentration and volume of material. The run continued for 7 hours. At the end of the run, analysis of the mother liquor showed 59% conversion to SA, 2.4% to succinamic acid, and 2.9% to succinimide. Cooling the mother liquor would result in a liquid portion and a solid portion that would be substantially pure SA.

### Example 2

This example demonstrates the effect of solvents on ammonia evolution from aqueous DAS. Run 10 is the control experiment where no solvent is present.

The outer necks of a three neck 1-L round bottom flask were fitted with a thermometer and a stopper. The center neck was fitted with a five tray 1″ Oldershaw section.

The Oldershaw section was topped with a distillation head. An ice cooled 500 mL round bottom flask was used as the receiver for the distillation head. The 1-L round bottom flask was charged with distilled water, the solvent being tested, SA and concentrated ammonium hydroxide solution. The contents were stirred with a magnetic stirrer to dissolve all the solids. After the solids dissolved, the contents were heated with the heating mantle to distill 350 g of distillate. The distillate was collected in the ice cooled 500 mL round bottom flask. The pot temperature was recorded as the last drop of distillate was collected. The pot contents were allowed to cool to room temperature and the weight of the residue and weight of the distillate were recorded. The ammonia content of the distillate was then determined via titration. The results were recorded in Tables 1 and 2.

TABLE 1

| Run # | 1 | 2 | 3 | 4 | 5 |
|---|---|---|---|---|---|
| Name of Acid charged | Succinic | Succinic | Succinic | Succinic | Succinic |
| Wt Acid Charged (g) | 11.8 | 11.81 | 11.83 | 11.8 | 11.78 |
| Moles Acid Charged | 0.1 | 0.1 | 0.1 | 0.1 | 0.1 |
| Wt 28% NH3 Solution Charged (g) | 12.76 | 12.78 | 12.01 | 12.98 | 13.1 |
| Moles NH3 Charged | 0.21 | 0.21 | 0.2 | 0.215 | 0.217 |
| Name of Solvent | DMSO | DMF | NMP | sulfolane | triglyme |
| Wt Solvent Charged (g) | 400.9 | 400 | 400 | 400 | 400 |
| Wt Water Charged (g) | 400 | 400 | 400 | 400 | 401 |
| Wt Distillate (g) | 350.1 | 365.9 | 351.3 | 352.1 | 351.2 |
| Wt Residue (g) | 467.8 | 455 | 460.5 | 457.1 | 473 |
| % Mass Accountability | 99.1 | 99.6 | 98.5 | 98.1 | 99.8 |
| Wt % NH3 in distillate (titration) | 0.91 | 0.81 | 0.78 | 0.71 | 0.91 |
| Moles NH3 in distillate | 0.187 | 0.174 | 0.161 | 0.147 | 0.188 |
| % NH3 removed in Distillate | 89 | 83 | 81 | 66 | 86 |
| % First NH3 removed in Distillate | 100 | 100 | 100 | 100 | 100 |
| % Second NH3 removed in Distillate | 78 | 66 | 62 | 32 | 72 |
| Final Pot Temp (° C.) | 138 | 114 | 126 | 113 | 103 |
| Final DAS/MAS/SA ratio | 0/22/78 | 0/34/66 | 0/38/62 | 0/68/32 | 0/28/72 |

*PG is propylene glycol

TABLE 2

| Run # | 6 | 7 | 8 | 9 | 10 |
|---|---|---|---|---|---|
| Name of Acid charged | Succinic | Succinic | Succinic | Succinic | Succinic |
| Wt Acid Charged (g) | 11.84 | 11.81 | 11.8 | 11.81 | 11.8 |
| Moles Acid Charged | 0.1 | 0.1 | 0.1 | 0.1 | 0.1 |
| Wt 28% NH3 Solution | 12.11 | 12.11 | 12.1 | 12.15 | 12.1 |
| Charged (g) | | | | | |
| Moles NH3 Charged | 0.2 | 0.2 | 0.2 | 0.2 | 0.2 |
| Name of Solvent | Dowanol TPM | Tetraglyme | Tetra PentaEG | HeavyMe GlyEther | none |
| Wt Solvent Charged (g) | 400.1 | 400 | 400 | 400.1 | 0 |
| Wt Water Charged (g) | 400 | 400 | 400 | 400.1 | 800 |
| Wt Distillate (g) | 350 | 345 | 350 | 349 | 351 |
| Wt Residue (g) | 468.4 | 473.8 | 465 | 470.4 | 466 |
| % Mass Accountability | 99.3 | 99.4 | 98.9 | 99.4 | 99.2 |
| Wt % NH3 in distillate | 0.58 | 0.62 | 0.55 | 0.6 | 0.13 |
| (titration) | | | | | |
| Moles NH3 in distillate | 0.119 | 0.126 | 0.113 | 0.123 | 0.027 |
| % NH3 removed in Distillate | 60 | 63 | 57 | 62 | 13.4 |
| % First NH3 removed in | 100 | 100 | 100 | 100 | 27 |
| Distillate | | | | | |
| % Second NH3 removed in | 20 | 26 | 14 | 24 | 0 |
| Distillate | | | | | |
| Final Pot Temp (° C.) | 104 | 110 | 115 | 113 | 100 |
| Final DAS/MAS/SA ratio | 0/80/20 | 0/74/26 | 0/86/14 | 0/76/24 | 83/27/0 |

### Example 3

This example used a DAS-containing, clarified fermentation broth derived from a fermentation broth containing E. coli strain ATCC PTA-5132.

The initial fermentation broth was clarified, thereby resulting in a clarified fermentation broth containing about 4.5% DAS. That clarified broth was used to produce crystalline SA as follows. The broth was first concentrated to approximately 9% using an RO membrane and then subjected to distillation at atmospheric pressure to further concentrate the broth to around 40%.

The concentrated broth was used as the starting material for conversion of DAS to SA, carried out batchwise in a 300 ml Parr reactor. A 200 g portion of the solution was reacted at 200° C./200 psig for 11 hours. As the reaction proceeded, water vapor and ammonia liberated from the DAS were condensed and collected overhead. Condensate was collected at about 2 g/min, and makeup water was fed back to the system at approximately the same rate.

Multiple samples were taken throughout the experiment. Samples taken early in the reaction indicated the presence of succinamide, succinamic acid, and succinimide. However, these nitrogen-containing byproducts decreased throughout the experiment. Conversion to SA was observed to be 55% in the final bottoms sample. The final solution was concentrated by evaporation and cooled to 4° C. The resulting crystalline solids were isolated via vacuum filtration, washed with ice water and dried under vacuum. The product (7 g) was essentially pure SA as determined by HPLC.

### Example 4

A 500 mL round bottom flask was charged with 80 g of an aqueous 36% DAS solution and 80 g of triglyme. The flask was fitted with a 5 tray 1″ glass Oldershaw column section which was topped with a distillation head. An addition funnel containing 3300 g of water was also connected to the flask. The flask was stirred with a magnetic stirrer and heated with a heating mantel. The distillate was collected in an ice cooled receiver. When the distillate started coming over the water in the addition funnel was added to the flask at the same rate as the distillate was being taken. A total of 3313 g of distillate was taken. The distillate contained 4.4 g of ammonia, as determined by titration. This means 37% of the DAS was converted to SA with the rest being converted to MAS. The residue in the flask was then placed in an Erlenmeyer flask and cooled to −4° C. while stirring. After stirring for 30 minutes the slurry was filtered while cold yielding 7.1 g of solids. The solids were dissolved in 7.1 g of hot water and then cooled in an ice bath while stirring. The cold slurry was filtered and the solids dried in a vacuum oven at 100° C. for 2 hrs yielding 3.9 g of SA. HPLC analysis indicated that the solids were SA with 0.099% succinamic acid present.

### Example 5

A pressure distillation column was made using an 8 ft long 1.5″ 316 SS Schedule 40 pipe packed with 316 SS Propak packing The base of the column was equipped with an immersion heater to serve as a reboiler. Nitrogen was injected into the reboiler via a needle valve to pressure. The overhead of the column had a total take-off line which went to a 316 SS shell and tube condenser with a receiver. The receiver was equipped with a pressure gauge and a back pressure regulator. Material was removed from the overhead receiver via blowcasing through a needle valve. Preheated feed was injected into the column at the top of the packing via a pump along with a dilute 0.4% sodium hydroxide solution. Preheated water was also injected into the reboiler via a pump. This column was first operated at 50 psig pressure which gave a column temperature of 150° C. The top of the column was fed a 4.7% DAS containing broth at a rate of 8 mL/min along with 0.15 mL/min of 0.4% sodium hydroxide solution. Water was fed to the reboiler at a rate of 4 mL/min. The overhead distillate rate was taken at 8 mL/min and the residue rate was taken at 4 mL/min. A total of 2565 g of broth was fed to the column along with 53 g of 0.4% sodium hydroxide solution. A total of 2750 g of distillate was taken and 1269 g of residue taken during the run. Titration of the distillate indicated that 71% of the total ammonia contained in the DAS was removed (i.e. the residue was a 42/58 mixture of SA/MAS). The composite residue was then fed back to the same column the next day under the following conditions; pressure 100 psig and temperature 173° C. The composite residue was fed to the top of the column at 4 mL/min along with 0.15 mL/min of 0.4% sodium hydroxide solution. The reboiler was fed water at 9.2 mL/min. A total of 1240 g of residue from the previous day was fed to the column along with 58 g of sodium hydroxide solution and 2890 g of water. A total of 3183 g of distillate was taken along with 1132 g of residue during the run. Titration of the distillate revealed an additional 14% of the ammonia was removed, yielding a 70/30 mixture of SA/MAS in the residue.

Although our processes have been described in connection with specific steps and forms thereof, it will be appreciated that a wide variety of equivalents may be substituted for the specified elements and steps described herein without departing from the spirit and scope of this disclosure as described in the appended claims.

1. A process for making nitrogen containing compounds of SA comprising:
(a) providing a clarified DAS-containing fermentation broth;
(b) distilling the broth under super atmospheric pressure at a temperature of >100° C. to about 250° C. to form an overhead that comprises water and ammonia, and a liquid bottoms that comprises SA, and at least about 20 wt % water;
(c) cooling and/or evaporating the bottoms to attain a temperature and composition sufficient to cause the bottoms to separate into a liquid portion and a solid portion that is substantially pure SA;
(d) separating the solid portion from the liquid portion;
(e) (1) contacting at least as part of the solid portion with hydrogen and an ammonia source in the presence of at least one hydrogenation catalyst to produce DAB, (2) dehydrating at least part of the solid portion to produce SDN or (3) dehydrating at least part of the solid portion to produce DAM; and
(f) recovering the DAB, SDN or DAM.

2. A process for making nitrogen containing compounds of SA comprising:
(a) providing a clarified DAS-containing fermentation broth;
(b) adding an ammonia separating and/or water azeotroping solvent to the broth;
(c) distilling the broth at a temperature and pressure sufficient to form an overhead that comprises water and ammonia, and a liquid bottoms that comprises SA, and at least about 20 wt % water;
(d) cooling and/or evaporating, the bottoms to attain a temperature and composition sufficient to cause the bottoms to separate into a liquid portion and a solid portion that is substantially pure SA;
(e) separating the solid portion from the liquid portion;
(f) (1) contacting at least a part of the solid portion with hydrogen and an ammonia source in the presence of at least one hydrogenation catalyst to produce DAB, (2) dehydrating it least part of the solid portion to produce SDN or (3) dehydrating at least part of the solid portion to produce DAM; and
(g) recovering the DAB, SDN or DAM.

3. A process for making nitrogen containing compounds of SA comprising:
providing a clarified MAS-containing fermentation broth;
(b) distilling the broth under super atmospheric pressure at a temperature of >100° C. to about 250° C. to form an overhead that comprises water and ammonia, and a liquid bottoms that comprises SA, and at least about 20 wt % water;
(c) cooling and/or evaporating the bottoms to attain a temperature and composition sufficient to cause the bottoms to separate into a liquid portion and a solid portion that is substantially pure SA;
(d) separating the solid portion from the liquid portion;
(e) (1) contacting at least a part of the solid portion with hydrogen and an ammonia source in the presence of at least one hydrogenation catalyst to produce DAB, (2) dehydrating at least part of the solid portion to produce SDN or (3) dehydrating at least part of the solid portion to produce DAM; and
(f) recovering the DAB, SDN or DAM.

4. A process for making nitrogen containing compounds of SA comprising:
(a) providing a clarified MAS-containing fermentation broth;
(b) adding an ammonia separating and/or water azeotroping solvent to the broth;
(c) distilling the broth at a temperature and pressure sufficient to form an overhead that comprises water and ammonia, and a liquid bottoms that comprises SA, and at least about 20 wt % water;
(d) cooling and/or evaporating the bottoms to attain a temperature and composition sufficient to cause the bottoms to separate into a liquid portion and a solid portion that is substantially pure SA;
(e) separating the solid portion from the liquid portion;
(f) (1) contacting at least a part of the solid portion with hydrogen and an ammonia source in the presence of at least one hydrogenation catalyst to produce DAB. (2) dehydrating at least part of the solid portion to produce SDN or (3) dehydrating at least part of the solid portion to produce DAM; and
(g) recovering the DAB, SDN or DAM.

5. The process of claim 1, wherein distilling the broth is carried out in the presence of an ammonia separating solvent which is at least one selected from the group consisting of diglyme, triglyme, tetraglyme sulfoxides, amides, sulfones, polyethyleneglycol (PEG), butoxytriglycol, N-methylpyrolidone (NMP), ethers, and methyl ethyl ketone (MEK) or in the presence of a water azeotroping solvent which is at least one selected from the group consisting of toluene, xylene, methylcyclohexane, methyl isobutyl ketone, hexane, cyclohexane and heptane.

6. The processes of claim 1, further comprising polymerizing the DAB with a dicarboxylic acid or ester to form a polyamide.

7. The processes of claim 1, further comprising contacting the SDN with hydrogen and ammonia in the presence of a hydrogenation catalyst to produce DAB.

8. The process of claim 7, further comprising polymerizing the DAB with a dicarboxylic acid or ester to form a polyamide.

9. The processes of claim 1, further comprising contacting the SDN with hydrogen and ammonia in the presence of a hydrogenation catalyst to produce a composition comprising SAN.

10. The process of claim 9, further comprising polymerizing the SAN to form a polyamide.

11. The process of claim 9, further comprising contacting the SAN with hydrogen and ammonia in the presence of a hydrogenation catalyst to produce DAB.

12. The process of claim 11, further comprising polymerizing the DAB with a dicarboxylic acid or ester to form a polyamide.

13. The processes of claim 1, further comprising dehydrating the DAM to produce SDN.

14. The process of claim 13, further comprising contacting the SDN with hydrogen and ammonia in the presence of a hydrogenation catalyst to produce DAB.

15. The process of claim 14, further comprising polymerizing the DAB with a dicarboxylic acid or ester to form a polyamide.

16. The process of claim 13, further comprising contacting the SDN with hydrogen and ammonia in the presence of a hydrogenation catalyst to produce a composition comprising SAN.

17. The process of claim 16, further comprising polymerizing the SAN to form a polyamide.

18. The process of claim 16, further comprising contacting the SAN with hydrogen and ammonia in the presence of a hydrogenation catalyst to produce DAB.

19. The process of claim 18 further comprising polymerizing the DAB with a dicarboxylic acid or ester to form a polyamide.
